(19) United States
(12) Patent Application Publication
(10) Pub. No.: US 2002/0123624 A1
(43) Pub. Date: Sep. 5, 2002

(54) Hydrophobically esterified starch products and process of making the same

(76) Inventors: Lei Qiao, Downingtown, PA (US); Qu-Ming Gu, Hockessin, DE (US); H. N. Cheng, Wilmington, DE (US)

Correspondence Address: Joanne Mary Fobare Rossi, Law Department, Hercules Incorporated, Hercules Plaza, Wilmington, DE, 19894-0001, US

(21) Appl. No.: 09/791,450

(22) Filed: Feb. 23, 2001

(51) Int. Cl. C12P 19/04; C08B 31/02

(52) U.S. Cl. 536/110; 435/101

(57) ABSTRACT

A hydrophobically esterified starch, is disclosed, having the general formula:

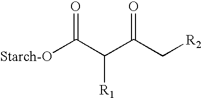

wherein R₁ and R₂ are separately linear or branched, saturated or unsaturated aliphatic chains having 1 to 22 carbons. Also disclosed is the process of making such hydrophobically esterified starch. The process can be enzymatic where an enzyme is used as a catalyst for the reaction. The process can also be chemical where no enzyme is used.

### BACKGROUND OF THE INVENTION

1. Field of the Invention

The present invention relates to the esterification of starch with a ketene dimer using enzymatic and chemical methods.

2. Description of Background and Related Art

Hydrophobically modified cellulose esters have been prepared by various chemical methods using carboxyl anhydrides. (C. J. Malta, Anal. Chem., 25(2), 245-249, 1953; C. J. Malta, Industrial and Engineering Chemistry 49(1), 84-88, 1957). Different types of mixed monocarboxyl esters of cellulose and cellulose ethers have been chemically synthesized, such as cellulose acetate succinate (J of Pharm. Sci. 51, 484, 1962) and hydroxypropyl cellulose acetate succinate (EP 0219426).

U.S. patent application Ser. No. 09/564,575, filed on May 5, 2000, discloses a novel process of making an esterified polysaccharide product using enzyme as a catalyst. It discloses that the reaction between alkyl ketene dimers and cellulosic or guar derivatives to make hydrophobically modified polysaccharide can be catalyzed by enzymes with high efficiencies.

Hydrophobically modified starches have been prepared by reactions of starch with 1) fatty acid chloride (JP 01054001 and EP 0859012), 2) fatty acid anhydride (U.S. Pat. No. 5,672,699, U.S. Pat. No. 6,037,466, U.S. Pat. No. 5,731,430). Although it is known for some compositions to contain both ketene dimers and starches (U.S. Pat. No. 4,861,376, U.S. Pat. No. 4,919,724, U.S. Pat. No. 5,190,584, WO 99/06219, and WO 99/54548), ketene dimers have generally not been used to react with starch to intentionally make the hydrophobically esterified starch products, either enzymatically or chemically.

Ketene dimers have an activated β-lactone functionality that reacts with hydroxy or amino groups under mild reaction conditions, which avoid the use of an acyl chloride or anhydride. Ketene dimers are inexpensive and few or no by-products are produced after the reaction.

Therefore, it would be advantageous to use ketene dimers as one of the starting materials to make hydrophobically esterified starches.

### SUMMARY OF THE INVENTION

The present invention provides a novel hydrophobically esterified starch product having the general formula:

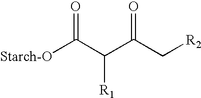

wherein R₁ and R₂ are separately linear or branched, saturated or unsaturated aliphatic chains having 1 to 22 carbons; in combination with an enzyme which in active form can catalyze the formation of the hydrophobically esterified starch.

The present invention is a reaction product between a starch and a ketene dimer. The product contains enzyme in deactivated form.

The enzyme used to catalyze the reaction, in active form, is obtained from animal, plant, bacteria, virus, yeast, fungi, or mixtures thereof. The enzyme can be a hydrolase.

The present invention also provides for a novel process of making a hydrophobically esterified starch by adding an effective amount of enzyme to a starch reaction mixture to catalyze the reaction. The starch reaction mixture comprises starch and ketene dimer. An effective amount of enzyme is about 0.01 to 30 wt. % based on the weight of the starch reaction mixture

The enzyme in this process can be a hydrolase. Specifically, the hydrolase is a lipase, esterase, or a protease obtained from animal, plant, bacteria, virus, fungi or mixtures thereof.

The starch used in the present novel process can be selected from the group consisting of maltodextrin, cyclodextrin, dextrin, amylose, amylopectin, cationic starch, anionic starch, oxidized starch, modified starch, and pre-gelatinised starch.

The ketene dimer used in the present process is one of alkyl ketene dimer, alkenyl ketene dimer, and ketene dimer of mixed fatty acids.

This invention also provides for a novel process of chemically making a hydrophobically esterified starch by reacting starch with ketene dimer.

### BRIEF DESCRIPTION OF THE FIGURES

FIG. 1 illustrates the effect of enzyme catalysis on the degree of substitution of the product; and

FIG. 2 illustrates the relationship between the product's degree of substitution and the product's viscosity.

### DETAILED DESCRIPTION OF THE INVENTION

As used herein:

The “viscosity” is measured using a DV-I Viscometer (Brookfield Viscosity Lab, Middleboro, Mass.). A selected spindle (number 2) is attached to the instrument, which is set for a speed of 30 RPM. A suspension of 4 weight % of starch in distilled water is heated at 90° C. for 30 min. The resulting mixture is cooled to room temperature. The Brookfield viscosity spindle is carefully inserted to the solution so as not to trap any air bubbles and then rotated at the above-mentioned speed for 3 minutes at 24° C. The units are centipoises.

The term “active form of enzyme” refers to those enzyme with active sites that, when combined with reactants, can catalyze the formation of a product from one or more than one reactants. The term “deactivating the enzyme” refers to eliminating the catalytic activity of the enzyme, such as by heating, for example.

The term “degree of substitution” means the average numbers of unit of X molecules, replacing OH and attached to each anhydroglucose unit, wherein X is defined as:

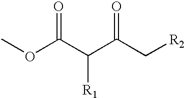

wherein R₁ and R₂ are separately linear or branched, saturated or unsaturated aliphatic chains having 1 to 22 carbons. R₁ and R₂ can also be separately linear or branched, saturated or unsaturated aliphatic chains having 1 to 16 carbons.

1. Enzymatic Synthesis of Hydrophobically Esterified Starch

One aspect of the present invention is a hydrophobically esterified starch which can be represented by the general structure as shown below:

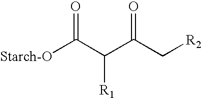

wherein R₁ and R₂ are separately linear or branched, saturated or unsaturated aliphatic chains having 1 to 22 carbons. In another embodiment of the invention, R₁ and R₂ are separately linear or branched, saturated or unsaturated aliphatic chains having 1 to 16 carbons. The esterification of starch by a ketene dimer causes the starch to become more hydrophobic and have higher viscosities and potentially emulsifying properties.

In a typical synthesis of the hydrophobically esterified starch, the starch, which constitutes about 0.1to 99 wt. % of the reaction mixture, is dissolved or suspended in an organic solvent containing a ketene dimer. The preferred amount of the starch is about 0.1 to 20 wt. % of the reaction mixture. The most preferred starch amount is about 0.1 to 10 wt. %. The ketene dimer is present in an amount of about 0.01 to no more than 90 wt. % of the reaction mixture. The preferred ketene dimer amount is about 0.01 to 50 wt. %. The most preferred ketene dimer amount is about 0.03 to 8 wt. %.

An enzyme, in its active form, is added to the reaction mixture to initiate the reaction. The amount of enzyme used is about 0.01 to 30 wt. % of the reaction mixture. The preferred enzyme amount is about 0.1 to 10 wt. %. The most preferred enzyme amount is about 1 to 5 wt. %. Without wishing to be bound by theory, it is presumed that the enzyme opens the β-lactone ring of the ketene dimer and forms a covalent intermediate (“acyl-enzyme intermediate”), which further reacts with the hydroxyl groups of the starch to form a β-keto ester of starch. Thus, ketene dimers are grafted onto the starch.

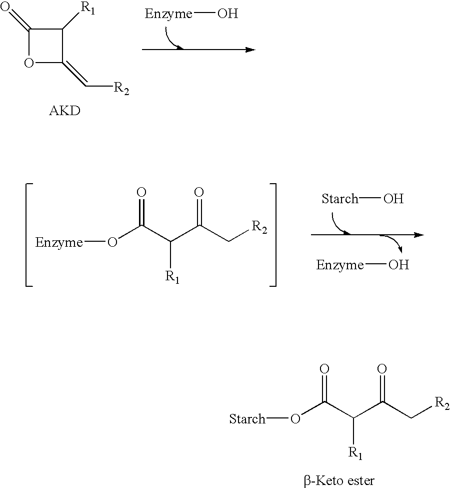

The use of enzyme accelerates the reaction. The reaction temperature is also lower compared to the reaction without using enzyme. The reaction time is about 0.25 to 72 hours. The preferable time is about 0.25 to 24 hours. The most preferred reaction time is 0.25 to 4 hours. The temperature of the reaction is preferably between about 5 to 100° C., more preferably between about 10 to 75° C., and most preferably between about 20 to 60° C.

At the end of the reaction, enzymes are deactivated using heat. The product can be precipitated by an organic solvent, such as isopropyl alcohol, acetone, or other similar organic solvents. A wash step can follow the precipitation step. The precipitated product is then washed with an organic solvent; examples include, but are not limited to, methylene chloride, chloroform, hexane, ethyl acetate, acetone or other similar organic solvents. Soxhlet extraction can be performed on the washed product with acetone, ethyl acetate, hexane, chloroform, methylene chloride or other similar organic solvents. The product can then be dried under vacuum.

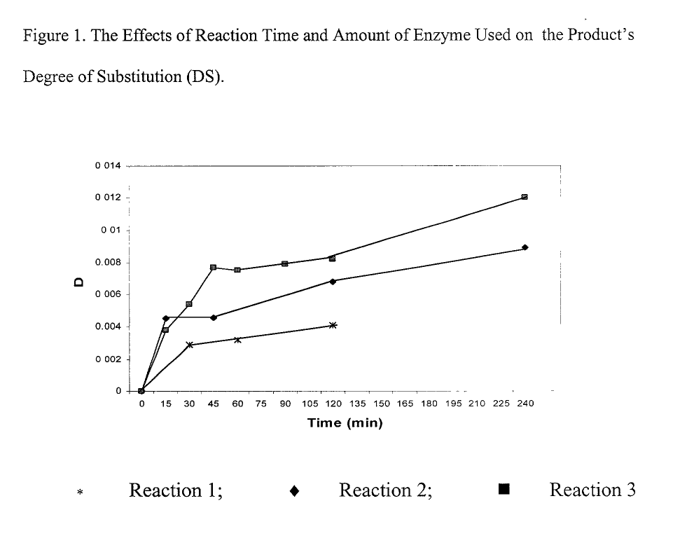

It is found that the degree of substitution of the hydrophobically esterified starch product ranges from about 0.0001 to about 0.027, preferably about 0.001 to 0.027, and most preferably about 0.003 to 0.027. The degree of substitution of the product of the present invention can be controlled by varying the reaction conditions such as the amount of enzyme used, the reaction time, temperature and pH, the concentrations of the starting materials such as starch or ketene dimers, and the reaction solvents. For instance, FIG. 1 illustrates the degree of substitution of the product as a function of reaction time. At 50 ° C., ketene dimer reacts with starch in dimethylsulfoxide (DMSO) to yield a low degree of substitution (DS) starch product (Reaction 1). In the presence of 8 wt. % of lipase PS based on the weight of the starch, the reaction proceeds more quickly and yields a product with a higher degree of substitution (Reaction 2). When more enzyme is used, such as 24 wt. % based on the weight of the starch, the reaction proceeds even faster and yields a product with an even higher degree of substitution (Reaction 3).

The hydrophobically esterified starch of the present invention has about 0.03 wt. % to no more than 90 wt. % of ketene dimer based on the total weight of the product. More preferably, the product has about 0.3 to 8.1 wt. % of ketene dimer. Most preferably, the product has about 1.0 to 8.1 wt. % of ketene dimer.

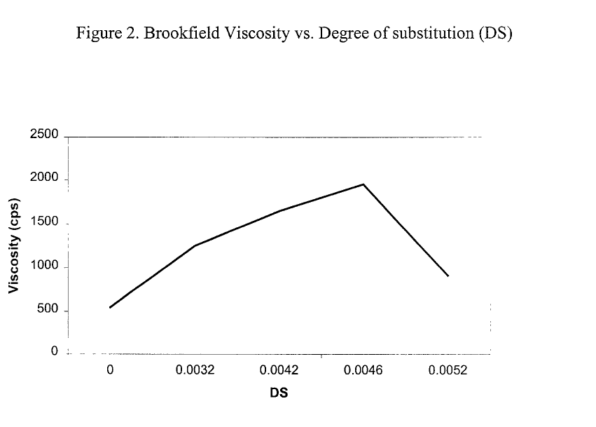

It is found that the viscosity of the hydrophobically esterified starch of the present invention is higher than that of the starting material at the same concentration. The viscosity of the product remains substantially unchanged for at least three days when the product is stored at pH of about 6.5 to 8.5. It is also found that as the degree of substitution of the product increases, the viscosity of the product increases initially and then decreases, presumably due to the decreased solubility. This is illustrated in FIG. 2.

Many different starches can be used in this invention. These include, but are not limited to, maltodextrin, cyclodextrin, dextrin, amylose, amylopectin, cationic starch, anionic starch, oxidized starch, modified starch, and pregelatinised starch. The preferred starches are from corn, potato, rice, wheat, and tapioca. The starches are present in an amount of about 0.1 to 99 wt. % of the reaction mixture. The preferred amount of starches is 0.1 to 20 wt. % of the reaction mixture. The most preferred amount of starches is 0.1 to 10 wt. % of the reaction mixture.

Many different ketene dimers can be used in this reaction. These include alkyl ketene dimers (AKD) such as Aquapel® from Hercules Incorporated (Wilmington, Del.), alkenyl ketene dimer such as Precis® from Hercules Incorporated (Wilmington, Del.), and various ketene dimers of mixed fatty acids. The preferred ketene dimers are aliphatyl or olefinyl ketene dimer with 6 to 36 carbons and 0 to 2 double bonds. More preferred ketene dimers are those wherein the alkyl or alkenyl groups are selected from stearyl, palmityl, oleyl, linoleyl groups and mixtures thereof. Some of the fatty acid ketene dimers are known materials, which are prepared by the 2+2 addition reaction of the alkyl ketenes.

Suitable solvent mediums include dimethylsulfoxide (DMSO), N,N-dimethylformamide (DMF), N,N-dimethylacetamide (DMAc), t-butyl methyl ether, and heptane. The solvent mediums are present in an amount of about 0 to 99.89 wt. % of the total reaction mixture. The preferred amount of the solvent mediums is about 0 to 95 wt. % of the reaction mixture. The most preferred amount of the solvent mediums is about 0 to 90 wt. % of the reaction mixture. These organic solvent mediums can also be used in combination with trace amount of water. Although organic solvent mediums help the reaction to be carried out more efficiently, the reaction can also proceed without the use of any solvent.

The process uses an enzyme, preferably lipase, under mild conditions in organic solvents. The enzyme, in active form, is used as a catalyst for the reaction of starch and ketene dimers. The enzyme used is preferably a hydrolase. More preferably it is a lipase, esterase, or protease. Such enzymes can be obtained from animal, plant, bacteria, virus, yeast, fungi, or mixtures thereof. Preferably the enzyme is obtained from Pseudomona sp., Pseudomona lipase, porcine pancreatic lipase, subtilisin or mixtures thereof. Most preferably, the enzyme is a lipase obtained from Pseudomonas cepacia. Similar enzyme from a synthetic source (e.g., protein synthesizer) may also be used.

Without further elaboration, it is believed that one skilled in the art can, using the preceding description, utilize the present invention to its fullest extent.

The following specific embodiments are, therefore, to be construed as merely illustrative, and not limitative of the remainder of the disclosure in any way whatsoever. In the following examples, all temperatures are set forth in degrees Celsius; unless otherwise indicated, all parts and percentages are by weight, unless otherwise indicated.

### EXAMPLE 1

Amylose (5.0 grams) and Aquapel® 364 (from Hercules Incorporated, 0.8 grams) are suspended in DMAc (100 ml). Lipase PS (from Amano, 0.4 grams) is added. The reaction mixture is incubated at 50° C. for 8 hours. The reaction mixture is then poured into isopropyl alcohol to precipitate the product. The precipitate is washed successively with methylene chloride and hexane before being dried at 50° C. under vacuum to a consistent weight.

¹H-NMR (DMSO-d6) δ 5.6 (s), 5.4 (s), 5.1 (s), 4.55 (s), 3.80-3.30 (m) 1.45 (m), 1.25 (s), 0.8 (t). The DS is determined to be 0.0057.

### EXAMPLE 2

This example illustrates the kinetics of the reaction between ketene dimer and starch. Cationic starch 52A (from National Starch, 5.0 grams) is dissolved in DMSO (100 ml) under mild heating. At about 50° C., Precis® 787 (from Hercules Incorporated, 1.6 grams) is added. Lipase PS (0.4 grams) is then added to the reaction mixture. The mixture is then incubated at 50° C. Aliquots of samples are taken at 0.25 h, 0.75 h, 2 h, 4 h, and 24 h. All samples are precipitated with isopropyl alcohol, washed with chloroform 3-4 times, and dried under vacuum. The degree of substitution of these samples is determined by ¹H-NMR to be 0.0045, 0.0046, 0.0068, 0.0089, and 0.0086, respectively.

The following examples 3-6 illustrate the effect of reactant concentration and amount of enzyme on the product's DS, and the relationship between DS and product viscosity.

### EXAMPLE 3

Cationic starch (Stalok 169 from Staley, 20 grams) is dissolved in DMSO (500 ml). Precis® 787 (0.8 grams) and lipase PS (1.6 grams) are added. The reaction mixture is incubated at 50° C. for 2 h. The reaction mixture is then precipitated with acetone and the precipitates are collected by filtration. The solid is then purified by Soxhlet extraction with isopropyl alcohol for 6 h. and then with acetone for 8 h. The solids are then dried at 50° C. under vacuum.

The degree of substitution of the product is determined by ¹H-NMR to be 0.0032. The Brookfield viscosity of the product sample in water at 4% concentration is 1256 cps. The unmodified starch, under the same measurement condition, has a viscosity of 546 cps.

### EXAMPLE 4

Example 3 is repeated under substantially identical condition except using 1.6 grams of Precis® 787.

The degree of substitution of the product is determined by ¹H-NMR to be 0.0042. The Brookfield viscosity of the product sample in water at 4% concentration is 1648 cps. The unmodified starch, under the same measurement condition, has a viscosity of 546 cps.

### EXAMPLE 5

Example 3 is repeated under substantially identical condition except using 3.2 grams of Precis® 787 and 2.4 grams of lipase PS.

The degree of substitution of the product is determined by ¹H-NMR to be 0.0046. The Brookfield viscosity of the product sample in water at 4% concentration is 1948 cps. The unmodified starch, under the same measurement condition, has a viscosity of 546 cps.

### EXAMPLE 6

Example 3 is repeated under substantially identical condition except using 3.2 grams of Precis® 787.

The degree of substitution of the product is determined by ¹H-NMR to be 0.0052. The Brookfield viscosity of the product sample in water at 4% concentration is 912 cps. The unmodified starch, under the same measurement condition, has a viscosity of 546 cps.

2. Chemical Synthesis of Hydrophobically Esterified Starch

The disclosed hydrophobically esterified starches of the present invention can also be made chemically without the presence of an enzyme as a catalyst. However, the efficiency of an uncatalyzed process is not as high as the catalyzed reaction. Compared with the enzymatic method, the same starting material selections and processes apply to the chemical method with the differences highlighted below.

In the reaction mixture, the weight ratio at the start of the reaction between ketene dimer and starch is about 0.01 to 5.0. The preferred ratio is about 0.1 to 3.5. At the end of the reaction, the product contains about 0.03 to 8 wt. % of ketene dimer. The reaction is usually performed by heating the reaction mixture at a temperature of about 50 to 120° C. for about 0.5 to 24 hours. Although the presence of organic solvent mediums helps the reaction to be carried out more efficiently, the reaction can also proceed without the use of any solvent.

The process can further comprise any one of more of the steps of precipitation, washing, extraction or drying under vacuum of the reaction product. At the end of the reaction, the reaction is neutralized with an acid and the product is precipitated by an organic solvent, such as isopropyl alcohol, acetone, or other similar organic solvents. The precipitated product is then washed with at least one of methylene chloride, chloroform, hexane, ethyl acetate, acetone or other similar organic solvents. Soxhlet extraction can be performed on the washed product with acetone, ethyl acetate, hexane, chloroform, methylene chloride or other similar organic solvents. The product is then dried under vacuum.

The chemical synthesis of hydrophobically esterified starch can be carried out in the presence or absence of a base. The chemical process can be catalyzed by a base. Both inorganic and organic bases can be used as the catalyst. Typically, sodium hydroxide can be used as an inorganic base, and 4,4-dimethylaminopyridine (DMAP) can be used as organic base. The reaction without a base present proceeds at a much slower rate.

Without further elaboration, it is believed that one skilled in the art can, using the preceding description, utilize the present invention to its fullest extent.

The following specific embodiments are, therefore, to be construed as merely illustrative, and not limitative of the remainder of the disclosure in any way whatsoever. In the following examples, all temperatures are set forth uncorrected in degrees Celsius; unless otherwise indicated, all parts and percentages are by weight.

### EXAMPLE 7

A mixture of cationic starch 52 (from National Starch, 2.5 grams), Aquapel® 364 (8.2 grams), and sodium hydroxide (0.6 grams) in DMSO (100 ml) is heated at about 110 -120° C. under an atmosphere of N₂ for 5 h. The reaction mixture is cooled to room temperature and poured into a mixture of 200 ml of acetone and 0.93 ml of acetic acid to precipitate out the product. The precipitate is then washed three times with chloroform and dried under vacuum to generate 1.9 grams of yellowish product. The degree of substitution of the product is 0.016.

### EXAMPLE 8

This example illustrates that the chemical reaction between ketene dimer and starch can be carried out without the use of an organic solvent medium.

Aquapel® 364 (from Hercules Incorporated, 0.8 grams) is dissolved in CH₂Cl₂ (10 mL). The solution is added to cationic starch 52A (from National Starch, 5.0 grams). The CH₂Cl₂ is then removed with a rotary evaporator. The resulting solid is heated to about 85-90° C. for 24 hours. The solid is then washed with chloroform 3-4 times, and dried under vacuum to give a white powder.

The degree of substitution of the product is determined by ¹H-NMR to be 0.0025.

3. Applications

The hydrophobically esterified starch made using the present invention can be used as ingredients in many applications where high viscosity and hydrophobic interaction are the desired properties. Such applications comprise uses as a paint thickener, paint stabilizer, construction materials, sizing agent in paper making process, emulsion stabilizer, and emulsifier in personal care products. They can also find uses as antihalation coatings, as excipient for tablets, and many other similar applications. For instance, the hydrophobically esterified starch made from the present invention may be added to paint to replace ordinary paint thickener, and it is expected that it may be used effectively as a paint thickener with desired properties. Also, this product may be added to construction materials. It is expected that shear and tensile strength of the construction material may be noticeably improved. This product may also be added to solutions to make personal care products such as lotion and hand cream. It is expected that high viscosity and emulsification may be achieved in these personal care products and such properties remain substantially unchanged after months.

From the foregoing description, one skilled in the art can easily ascertain the essential characteristics of this invention, and without departing from the spirit and scope thereof, can make various changes and modifications of the invention to adapt it to various usages and conditions. For instance, in all examples, the products are dried under vacuum. But the product can also be dried under atmospheric pressure.

What is claimed is:

1. A composition comprising a hydrophobically esterified starch having the general formula: 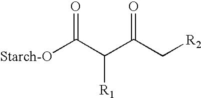
wherein R₁ and R₂ are separately linear or branched, saturated or unsaturated aliphatic chains having 1 to 22 carbons.

2. The composition of claim 1 wherein the hydrophobically esterified starch is in combination with an enzyme which in active form catalyzes the formation of said hydrophobically esterified starch.

3. The composition of claim 2 wherein said hydrophobically esterified starch is a reaction product between a starch and a ketene dimer.

4. The composition of claim 3 wherein said hydrophobically esterified starch composition comprises about 0.03 wt. % to no more than 90 wt. % of ketene dimer based on the total weight of said hydrophobically esterified starch product.

5. The composition according to claim 4 wherein said hydrophobically esterified starch product comprises about 0.3 wt. % to 8.1 wt. % of ketene dimer based on the total weight of said hydrophobically esterified starch product.

6. The composition according to claim 2 wherein said enzyme is a hydrolase.

7. The composition according to claim 6 wherein said hydrolase is a lipase, esterase, or a protease.

8. The composition according to claim 7 wherein said hydrolase is Pseudomonas lipase, porcine pancreatic lipase, or subtilisin.

9. The composition according to claim 7 wherein said hydrolase is a lipase selected from the group consisting of Pseudomonas sp., Pseudomonas cepacia and combinations thereof.

10. The composition according to claim 3 wherein said starch is at least one selected from the group consisting of maltodextrin, cyclodextrin, dextrin, amylose, amylopectin, cationic starch, anionic starch, oxidized starch, modified starch, and pre-gelatinised starch.

11. The composition according to claim 3 wherein said ketene dimer is at least one of (A) alkyl ketene dimer, (B) alkenyl ketene dimer, and (C) ketene dimer of mixed fatty acids.

12. The composition according to claim 3 wherein the degree of substitution of said reaction product ranges from about 0.0001 to about 0.027.

13. The composition according to claim 12 wherein the degree of substitution of said reaction product ranges from about 0.001 to about 0.027.

14. A composition comprising a hydrophobically esterified starch, which is an enzymatic reaction product between starch and ketene dimer, having the general formula: 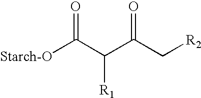
wherein R₁, and R₂ are separately linear or branched, saturated or unsaturated aliphatic chains having 1 to 22 carbons;
wherein said hydrophobically esterified starch comprises about 0.3 to 8.1 wt. % of ketene dimer based on the total weight of said hydrophobically esterified starch;
wherein said enzymatic reaction was catalyzed by a lipase from Pseudomonas cepacia;
wherein said ketene dimer is grafted onto said starch during the enzymatic reaction;
wherein said hydrophobically esterified starch has a higher viscosity compared to that of starch before reaction; and
wherein said hydrophobically esterified starch has a degree of substitution of about 0.001 to 0.027.

15. A process of making a hydrophobically esterified starch comprising adding an effective amount of enzyme to a starch reaction mixture to catalyze the reaction.

16. The process of claim 15 wherein said starch reaction mixture comprises starch and ketene dimer.

17. The process according to claim 15 wherein said effective amount of enzyme is about 0.01 to 30 wt. % based on the weight of said starch reaction mixture.

18. The process according to claim 17 wherein said effective amount of enzyme is about 1 to 5 wt. % based on the weight of said starch reaction mixture.

19. The process according to claim 15 wherein said enzyme is a hydrolase.

20. The process according to claim 19 wherein said hydrolase is Pseudomonas lipase, porcine pancreatic lipase, or subtilisin.

21. The process according to claim 20 wherein said hydrolase is a lipase selected from the group consisting of Pseudomonas sp., Pseudomonas cepacia and combinations thereof.

22. The process according to claim 16 further comprises a reaction medium wherein said reaction medium comprises an organic solvent medium and said organic solvent medium is selected for the group consisting of DMSO, DMF, DMAc, t-butyl methyl ether, and heptane or combinations thereof.

23. The process according to claim 16 wherein said starch is at least one selected from the group consisting of maltodextrin, cyclodextrin, dextrin, amylose, amylopectin, cationic starch, anionic starch, oxidized starch, modified starch, and pre-gelatinised starch.

24. The process according to claim 23 wherein said starch is present in an amount of about 0.1 to 99 wt. % based on the total weight of the reaction mixture.

25. The process according to claim 24 wherein said starch is present in an amount of about 0.1 to 20 wt. % based on the total weight of the reaction mixture.

26. The process according to claim 16 wherein said ketene dimer is one of alkyl ketene dimer, alkenyl dimer, and ketene dimer of mixed fatty acids.

27. The process according to claim 26 wherein said ketene dimer is present in an amount of about 0.01 wt. % to no more than 90 wt. % based on the total weight of the reaction mixture.

28. The process according to claim 26 wherein said ketene dimer is present in an amount of about 0.03 to 8.1 wt. % based on the total weight of the reaction mixture.

29. The process according to claim 15 wherein the reaction time is between about 0.25 to 72 hours.

30. The process according to claim 15 comprising deactivating the enzyme by the end of the reaction.

31. The process according to claim 30 further comprising one or more steps selected from the group consisting of precipitating the reaction product with isopropyl alcohol or acetone, washing the reaction product with organic solvents; said organic solvents being at least one of methylene chloride, chloroform, hexane, ethyl acetate, and acetone, performing a Soxhlet extraction on the reaction product with an organic solvent; said organic solvent being at least one of acetone, ethyl acetate, hexane, chloroform, or methylene chloride, drying the reaction product under vacuum.

32. A process of making a hydrophobically esterified starch comprising:
adding enzyme to a reaction mixture in the amount of about 1 to 5 wt. % based on the weight of said reaction mixture;
said reaction mixture comprising:
(A) a starch in an amount of about 0.1 to 10 wt. % based on the total weight of said reaction mixture;
(B) a ketene dimer in an amount of about 0.03 to 8.1 wt. % based on the total weight of said reaction mixture; and
(C) an organic solvent present in the amount of about 0 to 90 wt. % based on the total weight of said reaction mixture;
said enzyme comprising a lipase from Pseudomonas cepacia;
heating the reaction mixture for about 0.25 to 8 hours at about 20 to 60° C.;
deactivating said enzyme by the end of the reaction;
precipitating the reaction product with isopropyl alcohol or acetone;
washing the reaction product with an organic solvent;
extracting the reaction product with an organic solvent; and
drying the reaction product under vacuum.

33. A process of chemically making a hydrophobically esterified starch comprising reacting starch with ketene dimer.
